(19) United States
(12) Patent Application Publication
(10) Pub. No.: US 2026/0090913 A1
(43) Pub. Date: Apr. 2, 2026

(54) IMPLANT INSERTION DEVICE FOR PUNCTA AND OTHER ORIFICES

(71) Applicant: MLMJO, LLC, Bettendorf, IA (US)

(72) Inventor: Martin O'Malley, Bettendorf, IA (US)

(21) Appl. No.: 19/338,201

(22) Filed: Sep. 24, 2025

Related U.S. Application Data
(60) Provisional application No. 63/699,883, filed on Sep. 27, 2024.

(51) Int. Cl. A61F 9/007

(52) U.S. Cl. CPC A61F 9/00772

(57) ABSTRACT

An insertion device is provided for implant or other payload delivery to canal of a patient or other subject via an orifice, for example, a punctum. The insertion device may include a barrel, barrel loading interface, tip, plunger, implant and barrel alignment pathways and corresponding plunger alignment guides. In some embodiments the plunger includes a central plunger and stabilizing plungers. A method for implant or other payload delivery to canal of a patient or other subject using the insertion device is also provided.

### CROSS-REFERENCE TO RELATED APPLICATION

This application claims the priority from U.S. provisional patent application serial number 63/699,883 filed September 27, 2024. The foregoing application is incorporated in its entirety herein by reference.

### FIELD OF THE INVENTION

The present disclosure relates to an orifice insertion device. More particularly, the disclosure relates to implant or other payload delivery to canal of a patient or other subject via an orifice such as a punctum.

### BACKGROUND

A punctal insertion device is a medical instrument designed for procedures involving the punctum, the tiny opening at the inner corner of the eyelid where tears drain. These devices can serve a range of purposes, including diagnosing, treating conditions related to tear drainage such as dry eye syndrome, and delivery of medication. Common types of orifice insertion devices used in the prior art include dilators to widen the punctum, implants to block tear drainage, and probes to examine the tear ducts.

However, such devices of the prior art undesirably require ensuring the punctum remains adequately dilated throughout the implant loading and delivery process. If the punctum constricts, it can impede the smooth insertion of the device and the implant, potentially causing discomfort, failure of proper function of the implant, or even tissue damage to the patient. Fumbling or mishandling the implant can lead to contamination, damage, or misplacement, compromising the effectiveness of the procedure.

Furthermore, achieving the correct depth of implant placement is essential for its optimal function and can present a challenge using devices in the prior art. Inserting an implant too shallowly might result in expulsion or discomfort, while placing it too deeply could cause irritation or complications. The placement procedure using devices of the prior art often necessitates a high degree of dexterity and hand-eye coordination, as the limited space and the need for precise movements can make the process challenging, especially for less experienced practitioners. For example, causing an implant to contact moisture prematurely could impact the performance of an implant or even render it unusable. Maintaining a dry environment during the loading and placement process is critical. No known orifice insertion device exists that facilitates accurate and consistent application of an implant into the canal of a patient with improved efficiency such as reducing the risk of patient discomfort, complications, and compromised outcomes.

Therefore, a need exists to solve the deficiencies present in the prior art. What is needed is an insertion device capable of efficiently delivering an implant to a desired canal. What is needed is an insertion device that can maintain dilation throughout implant loading and delivery, for example, to a desired placement depth. What is needed is an insertion device featuring improved handling and manipulation. What is needed is an insertion device configured to deliver an implant with sufficient depth and expulsion of the implant. What is needed is an insertion device operable without requiring preliminary dilation. What is needed is an insertion device to deliver an insert for improved ease of insertion with limited dexterity. What is needed is an insertion device to deliver an insert with decreased risk of premature hydration.

### SUMMARY

An aspect of the disclosure advantageously provides an insertion device capable of efficiently delivering an implant to a desired canal. An aspect of the disclosure advantageously provides an insertion device that can maintain dilation throughout implant loading and delivery, for example, to a desired placement depth. An aspect of the disclosure advantageously provides an insertion device featuring improved handling and manipulation. An aspect of the disclosure advantageously provides an insertion device configured to deliver an implant with sufficient depth and expulsion of the implant. An aspect of the disclosure advantageously provides an insertion device operable without requiring preliminary dilation. An aspect of the disclosure advantageously provides an insertion device to deliver an insert for improved ease of insertion with limited dexterity. An aspect of the disclosure advantageously provides an insertion device to deliver an insert with decreased risk of premature hydration.

An insertion device enabled by this disclosure may advantageously provide an ability to set a placement depth of an implant within a canal of a patient and pull encompassing tissue up and at least partially around the implant. The disclosure enables an orifice insertion device featuring a barrel with axial gaps at an engagement end. A plunger, which may be partially located within the barrel, can move through the barrel. The plunger may include a protruding portion extending beyond the barrel through the axial gaps. This protruding portion may serve as an alignment guide, which may encompass assisting with delivering an implant to a desired placement depth. In various embodiments, the plunger design and the barrel may incorporate additional features like a stabilizer tab and a loading platform for easy and secure loading of objects, such as implants, medications, or deliverable payloads, into the device.

Accordingly, the disclosure may feature an insertion device to deliver an implant to a canal of a patient via an orifice including a barrel and a plunger. The barrel may extend longitudinally from a barrel action end to a barrel engagement end. The plunger may extend longitudinally from a plunger action end to a plunger engagement end. The plunger may be configurable to receive the implant to be delivered. The plunger may selectively travel about the barrel upon application of an actuation force to move the implant toward the barrel engagement end. The implant may be selectively pushed by the plunger through the barrel engagement end to be operatively delivered to the canal. The implant is deliverable to the canal without requiring preliminary dilation of the orifice.

In another aspect, the barrel may include a barrel axial gap extending longitudinally inward from the barrel engagement end. The plunger may include a protruding portion that at least partially protrudes beyond a barrel circumference via the barrel axial gap.

In another aspect, the plunger engagement end may include a plunger delivery surface to distribute the actuation force provided via the plunger to an increased surface area of the implant.

In another aspect, the plunger delivery surface may extend outwardly from the barrel axial gap to selectively abut tissue adjacent to the orifice.

In another aspect, the plunger delivery surface may include segments of varied depth to deliver the implant to a selectable depth within the canal, such as a first delivery surface depth to engage with the implant such to apply a pushing force to deliver the implant to the canal at the selectable depth and a second delivery surface depth to interact with tissue adjacent to the orifice and effectively limit travel of the plunger.

In another aspect, a tip may be operatively installed to the engagement end of the barrel to selectively interface with the orifice during operation. The implant may pass through the tip from the barrel.

In another aspect, the tip may include a tip axial gap that reversibly expands upon passing of the implant through the tip to permit guided installation of the implant to the canal.

In another aspect, the barrel may include a barrel loading section located on a side of the barrel through which the implant is selectively loaded into the barrel, without limitation.

In another aspect, the plunger further may include a central plunger and a stabilizing plunger. The central plunger may engage with and selectively deliver the implant. The stabilizing plunger may be capable of moving independently from the central plunger to engage tissue adjacent to the orifice to provide stability during delivery of the implant and selectively configure a depth at which the implant is delivered into the canal.

In another aspect, the barrel may include barrel covers located at the barrel action end and the barrel engagement end. The central plunger may pass through the barrel via the barrel covers through a barrel cover central aperture. The stabilizing plunger may pass through the barrel via the barrel covers through barrel cover stabilizing apertures.

In another aspect, stabilizer feet may be installed to the stabilizing plunger at the plunger engagement end.

In another aspect, the stabilizing plunger may include at least two elongated stabilizing plunger members.

According to an embodiment enabled by this disclosure, an insertion device for interacting with an orifice of a body is provided including a barrel, tip, and plunger. The barrel may extend longitudinally from a barrel action end to a barrel engagement end and include a barrel axial gap extending longitudinally inward from the barrel engagement end. The tip may be operatively installed to the barrel engagement end. The plunger may extend longitudinally from a plunger action end to a plunger engagement end and include a protruding portion that at least partially protrudes beyond a barrel circumference via the barrel axial gap. The plunger may be configurable to receive the implant to be delivered. The plunger engagement end may include a plunger delivery surface to distribute the actuation force provided via the plunger to an increased surface area of the implant. The plunger may selectively travel about the barrel upon application of the actuation force to move the implant toward the barrel engagement end. The implant may be selectively pushed by the plunger through the barrel engagement end and the tip to be operatively delivered to a canal of a patient via the orifice.

In another aspect, the plunger delivery surface may extend outwardly from the barrel axial gap to selectively abut tissue adjacent to the orifice.

In another aspect, the tip may selectively interface with the orifice during operation. The implant is deliverable to the canal without requiring preliminary dilation of the orifice.

In another aspect, the barrel may further include a loading platform located approximately along a longitudinal edge of, and extending outwardly from, the barrel loading section.

According to an embodiment enabled by this disclosure, an insertion device to deliver an implant to a canal of a patient via an orifice is provided including a barrel and a plunger. The barrel may extend longitudinally from a barrel action end to a barrel engagement end. The plunger may extend longitudinally from a plunger action end to a plunger engagement end. The plunger may include a central plunger configurable to receive and selectively deliver the implant. The plunger may also include a stabilizing plunger capable of moving independently from the central plunger to engage tissue adjacent to the canal to provide stability during delivery of the implant and selectively configure a depth at which the implant is delivered. The plunger may selectively travel about the barrel upon application of an actuation force to move the implant toward the barrel engagement end. The implant may be deliverable to the canal without requiring preliminary dilation of the orifice.

In another aspect, the central plunger may include a plunger delivery surface to distribute the actuation force provided via the central plunger to an increased surface area of the implant.

In another aspect, stabilizer feet may be installed to the stabilizing plunger at the plunger engagement end to selectively abut the tissue.

In another aspect, the barrel may further include a barrel loading section located on a side of the barrel through which the implant is selectively loaded into the barrel.

In another aspect, a tip may be operatively installed to the barrel engagement end to selectively interface with the orifice, the implant selectively passing through the tip from the barrel.

Terms and expressions used throughout this disclosure are to be interpreted broadly. Terms are intended to be understood respective to the definitions provided by this specification. Technical dictionaries and common meanings understood within the applicable art are intended to supplement these definitions. In instances where no suitable definition can be determined from the specification or technical dictionaries, such terms should be understood according to their plain and common meaning. However, any definitions provided by the specification will govern above all other sources.

Various objects, features, aspects, and advantages described by this disclosure will become more apparent from the following detailed description, along with the accompanying drawings in which like numerals represent like components.

### BRIEF DESCRIPTION OF THE DRAWINGS

FIG. 1 is a perspective view of an illustrative insertion device in a preloded embodiment, according to an embodiment of this disclosure.

FIG. 2 is a top plan view of an illustrative insertion device of FIG. 1.

FIG. 3 is a perspective view of an illustrative barrel of the insertion device of FIG. 1, according to an embodiment of this disclosure.

FIG. 4 is a top plan view of the illustrative barrel of FIG. 3.

FIG. 5 is a perspective view of an illustrative plunger of the insertion device if FIG. 1, according to an embodiment of this disclosure.

FIG. 6 is a perspective view of an illustrative insertion device in a loadable embodiment, according to an embodiment of this disclosure.

FIG. 7 is a top plan view of the illustrative insertion device of FIG. 6.

FIG. 8 is a perspective view of an illustrative barrel of the insertion device of FIG. 6, according to an embodiment of this disclosure.

FIG. 9 is a side elevation view of an illustrative central plunger of a insertion device, according to an embodiment of this disclosure.

FIG. 10 is a perspective view of an illustrative stabilizing plunger, according to an embodiment of this disclosure.

FIG. 11 is a perspective view of the illustrative insertion device that is loadable and in a single plunger configuration, according to an embodiment of this disclosure.

FIG. 12 is a perspective view of an illustrative insertion device barrel comprising a loading section, according to an embodiment of this disclosure.

FIG. 13 is a perspective view of an illustrative insertion device approaching a canal, according to an embodiment of this disclosure.

FIG. 14 is a perspective view of an illustrative insertion device engaging a canal, according to an embodiment of this disclosure.

FIG. 15 is a perspective view of an illustrative insertion device deploying an insert into a canal, according to an embodiment of this disclosure.

FIG. 16 is a perspective view of an insert installed into a canal, according to an embodiment of this disclosure.

### DETAILED DESCRIPTION

The following disclosure is provided to describe various embodiments of an insertion device, such as a punctal insertion device, without limitation. Skilled artisans will appreciate additional embodiments and uses of the present invention that extend beyond the examples of this disclosure. Terms included by any claim are to be interpreted as defined within this disclosure. Singular forms should be read to contemplate and disclose plural alternatives. Similarly, plural forms should be read to contemplate and disclose singular alternatives. Conjunctions should be read as inclusive except where stated otherwise.

Expressions such as “at least one of A, B, and C” should be read to permit any of A, B, or C singularly or in combination with the remaining elements. Additionally, such groups may include multiple instances of one or more element in that group, which may be included with other elements of the group. All numbers, measurements, and values are given as approximations unless expressly stated otherwise.

For the purpose of clearly describing the components and features discussed throughout this disclosure, some frequently used terms will now be defined, without limitation. The term barrel, as it is used throughout this disclosure, is defined as a substantially cylindrical chamber or tube having an interior volume through which a plunger, implant, and/or other payload may pass. The term plunger, as it is used throughout this disclosure, is defined as a mechanism, often a rod or piston-like component, that applies force or pressure within the device to propel or push the implant from the barrel to a target canal and/or lumen. The term implant, as it is used throughout this disclosure, is defined as a small, specially designed object that is intended to be inserted into the canal of a patient, which can include canals related to the eyes.

The term canal, as it is used throughout this disclosure, is defined as a passage or channel within a patient's body, for example, a lacrimal canaliculi or other anatomical canal, capable of receiving an implant. The term orifice, as it is used throughout this disclosure, is defined as an opening on a body, such as the punctum at the inner corner of the eyelid, through which an implant may be delivered into a canal.

Various aspects of the present disclosure will now be described in detail, without limitation. In the following disclosure, a punctual insertion device will be discussed. Those of skill in the art will appreciate alternative labeling of the punctual insertion device as an implant placement apparatus, canal implant delivery system, punctual canal implanter, punctual syringe-based implant installation apparatus, the invention, or other similar names. Similarly, those of skill in the art will appreciate alternative labeling of the punctual insertion device as a method for canal implantation, punctual implant placement procedure, guided implant delivery method, method, operation, the invention, or other similar names. Skilled readers should not view the inclusion of any alternative labels as limiting in any way.

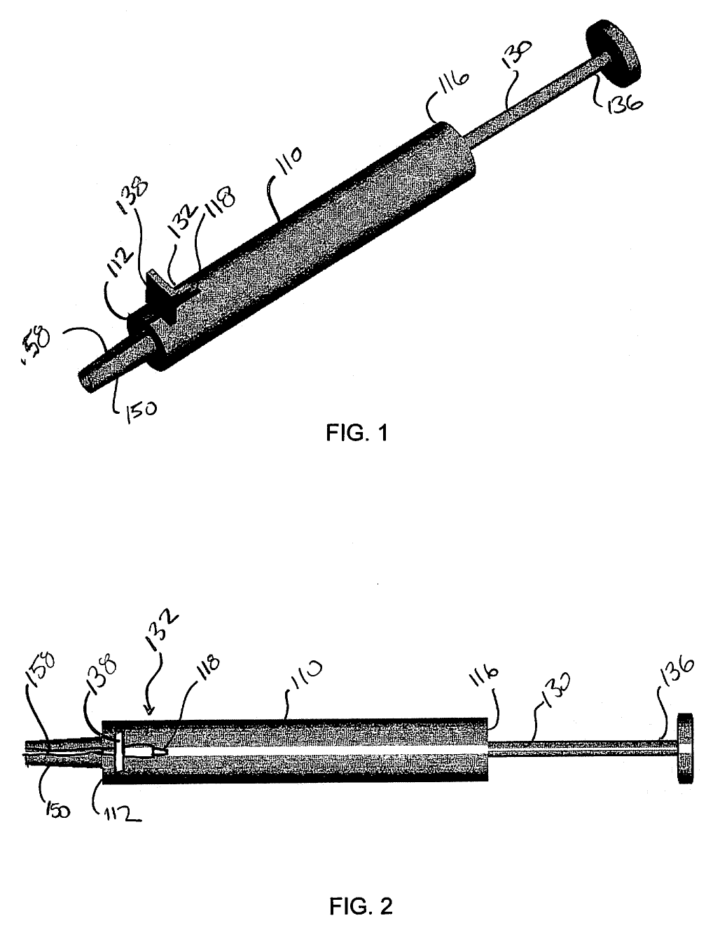

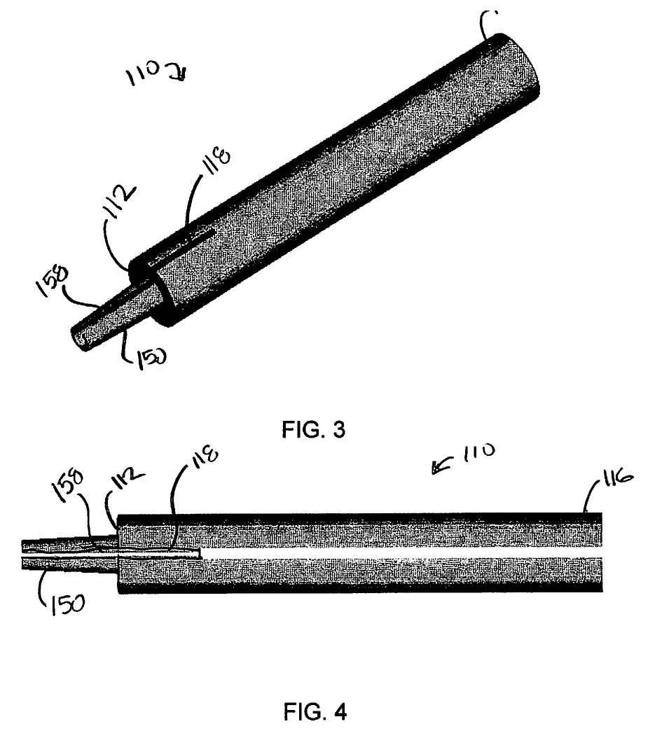

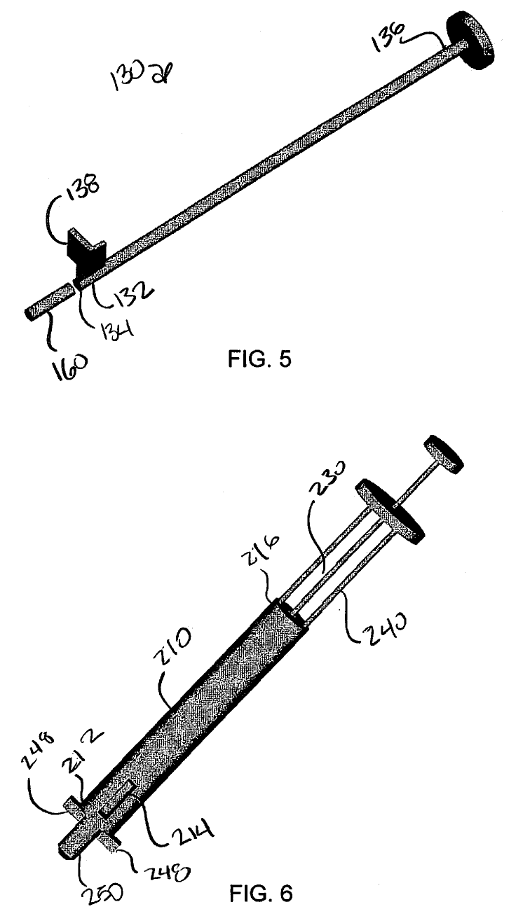

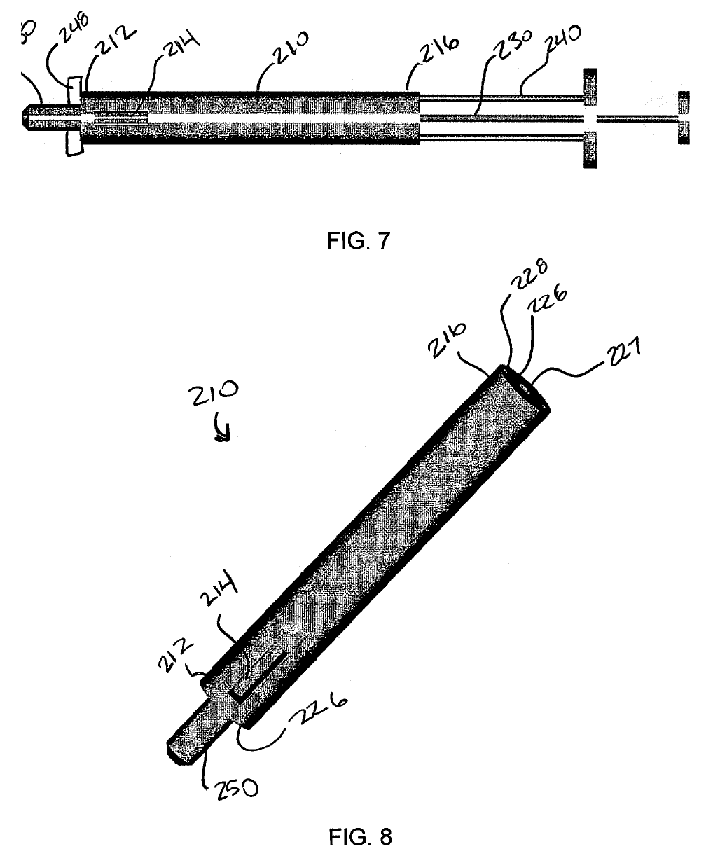

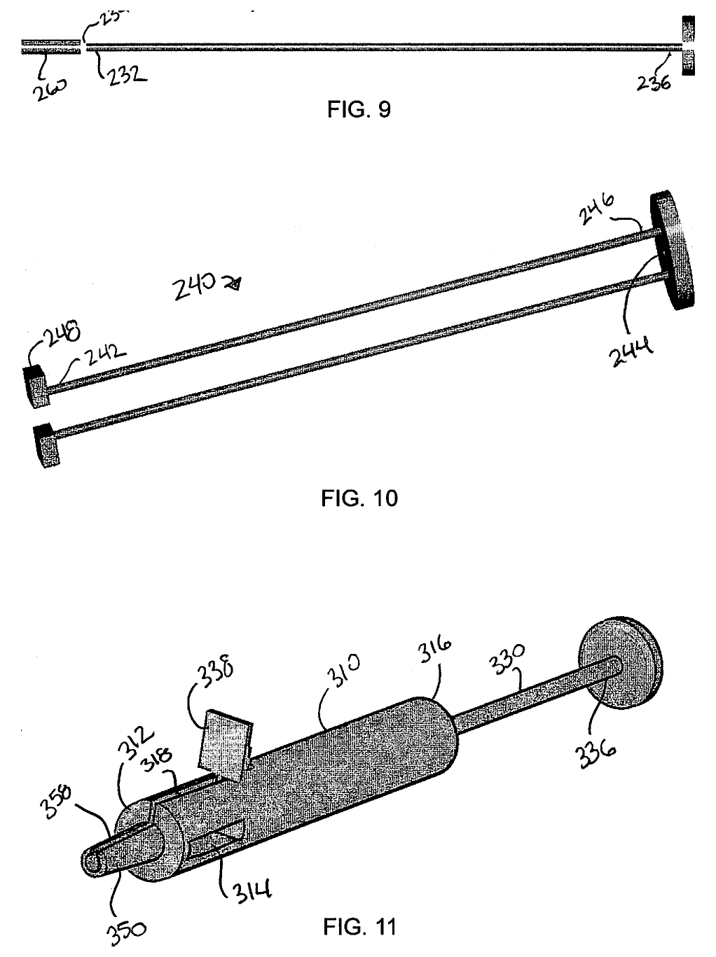

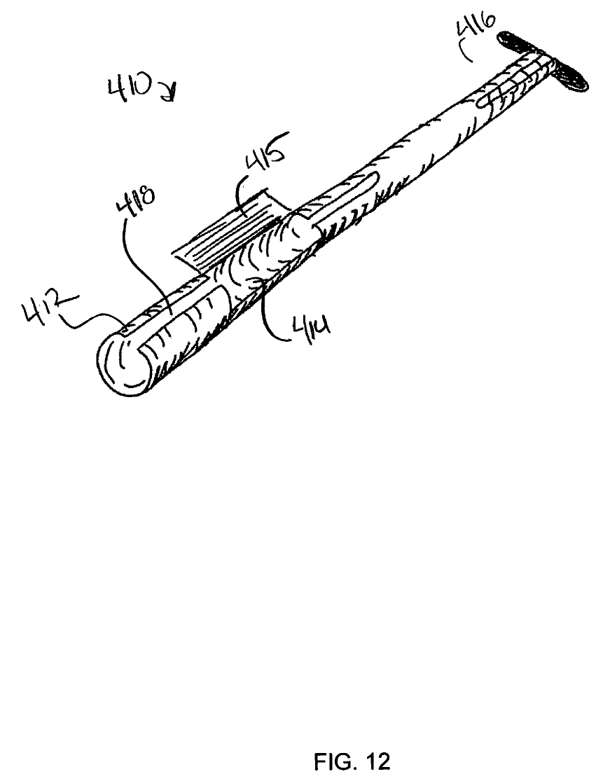

Referring now to FIGS. 1-16, the insertion device will now be discussed in more detail. The insertion device may include a barrel, barrel loading interface in some embodiments, tip, plunger, implant, barrel alignment pathways and corresponding plunger alignment guides, and additional components that will be discussed in greater detail below. The insertion device may operate one or more of these components interactively with other components for implant or other payload delivery to a canal of a patient or other subject. The insertion device may be configured in various embodiments, for example and without limitation, in a preloaded or loadable configuration.

The insertion device enabled by this disclosure may facilitate performing procedures on a canal of a patient, for example the canal being a punctum or tear duct opening at the inner corner of the eyelid. The insertion device may include a barrel 110, 210, 310, 410 extending from an engagement end, where the insertion device may interface with the punctum or another orifice to a canal, to a distal action end. This barrel 110, 210, 310, 410 may include axial gaps near the engagement end to assist with guiding and stabilizing the plunger 130 and implant 160, 260 during insertion. In certain embodiments, the engagement end of the barrel 110, 210, 310, 410 may include a tip 150, 250, 350 that is tapered, which may facilitate gentle and atraumatic insertion into the delicate punctal opening.

The barrel 110, 210, 310, 410 may also include a loading platform 415 in some embodiments for convenient and secure loading of the implant 160, 260 into the insertion device. In at least one embodiment, the loading platform 415 may be operatively attached to the barrel 110, 210, 310, 410 in a fixed location. In an alternative embodiment, one or more optional loading doors are provided to act as a loading platform 415 when in the open position and can be selectively closed to ensure the implant 160, 260 remains safely in place during the procedure.

A plunger 130, 230, 240, 330 which may be at least partially housed within the barrel 110, 210, 310, 410, may enable controlled movement of the implant 160, 260. The plunger may extend from a plunger delivery surface 134, 234 at a barrel engagement end 112, 212, 312, 412 to a distal barrel action end 116, 216, 316, 416, facilitating precise delivery. In one embodiment, a portion of the plunger 130, 330 may protrude beyond the barrel 110, 310, 410 through the axial gaps at the barrel engagement end 112, 312, 412 to advantageously assist in aligning the device, which may assist with delivering an implant 160, 260 to a desired placement depth, and potentially serve as a guide for an implant 160, 260. In some embodiments, the plunger may be provided as a central plunger 230 and a stabilizing plunger 240, which may pass through barrel caps operated independently to travel through the barrel 210.

The barrel will now be discussed in greater detail. FIGS. 1-4, 6-8, and 11-12 highlight examples of the barrel, which may also be shown in other figures. The barrel 110, 210, 310, 410 forms a tubular pathway through which the implant 160, 260 travels during the insertion process. The barrel 110, 210, 310, 410 is typically crafted using materials such as plastic, glass, stainless steel, or other materials that would be appreciated by a person of skill in the art to provide structural rigidity while also minimizing the risk of adverse reactions within the delicate ocular or other environment. The barrel 110, 210, 310, 410 may extend from an engagement end 112, 212, 312, 412 that interfaces with the punctal opening or other orifice along the length of the barrel 110, 210, 310, 410 to a distal action end 116, 216, 316, 416.

The barrel engagement end 112, 212, 312, 412 is the portion of the insertion device that may selectively interface with the orifice of a patient. It serves as the terminal point of a pathway through which an implant 160, 260 may travel during the insertion process. The barrel engagement end 112, 212, 312, 412 may guide and permit the passage of the implant 160, 260 as it is pushed by the plunger 130, 230, 330 out of the barrel 110, 210, 310, 410 and into the targeted canal. The design of the barrel engagement end 112, 212, 312, 412 may be adapted to accommodate different device configurations, including versions that are pre-loaded with an implant 160 as well as versions that allow a user to load the implant 260 before use.

The barrel engagement end 112, 312, 412 may include a barrel axial gap 118, 318, 418 which may be a channel or slot extending longitudinally inward from the end of the barrel 110, 310, 410. This gap may be configured to accommodate and guide a corresponding protruding portion 138, 338 of the plunger 130, 330 as it moves through the barrel 110, 310, 410. This interaction between the gap and the plunger 130, 330 may advantageously reduce the risk of the implant 160 being rotated or moved in an undesired way while it is being delivered into the canal of the patient. The circumferential border of the aperture at the barrel engagement end 112, 312, 412 may include openings corresponding with the barrel axial gap 118, 318, 418 allowing the plunger 130, 330 to pass at least partially through the aperture during administration of the implant 160.

In some embodiments, a tip 150, 250, 350 may be operatively installed to the barrel engagement end 112, 212, 312. In some embodiments, the tip 150, 250, 350 is installed to the engagement end 112, 212, 312 by being a continuation of material from the barrel 110, 210, 310 in one continuous piece. This tip 150, 250, 350 may be tapered, which may facilitate a gentle and atraumatic insertion into a delicate punctal opening. The tip 150, 250, 350 may also feature a tip axial gap 158, 358 that aligns with the barrel axial gap 118, 318, permitting the protruding portion 138, 338 of the plunger 130, 330 to continue traveling through the tip 150, 250, 350 for guided installation of the implant 160, 260. In at least one embodiment, the tip 150, 250, 350 may be constructed using an at least partially flexible material, which may allow the tip 150, 250, 350 to deform or reversibly expand as the implant 160, 260 is passed through it by the plunger 130.

For embodiments of the insertion device using a pre-loaded configuration, the implant 160 may already be contained within the delivery passage at or near the barrel engagement end 112. In such a configuration, a barrel loading section or access slot near the engagement end may not be present.

Conversely, for embodiments that allow loadable operation, for example via a non pre-loaded configuration, the insertion device may be characterized by a barrel loading section 214, 314, 414 located on a side of the barrel 210, 310, 410. This loading section 214, 314, 414, which may be an access window, allows for the placement of an implant 260 into the device before depressing the plunger. This loading feature is often located near the barrel engagement end 212, 312, 412 to promote easy access and secure placement of the implant 260 into the barrel 210, 310, 410 prior to operation. After loading, the plunger may be advanced to move the implant 260 along the barrel 210, 310, 410 until it is positioned at the barrel engagement end 212, 312 ready to be inserted into the canal.

In some embodiments, the insertion device may include barrel covers 226 located at the barrel action end 216 and the barrel engagement end 212. These components may serve to enclose the ends of the barrel 210 main body. The barrel covers 226 may be configured with openings to guide the components of a multi-part plunger 230, 240, ensuring that different parts of the plunger 230, 240 remain aligned as they travel through the barrel 210. This configuration may be particularly applicable to versions of the device that utilizes a separate central plunger 230 and stabilizing plunger 240.

The barrel covers 226 may each include a barrel cover central aperture 227. This central aperture is an opening through which the central plunger 230 may pass. By providing a dedicated pathway, the barrel cover central aperture 227 may help to maintain the alignment of the central plunger 230 as it moves longitudinally within the barrel 210. This guidance may assist in the precise and controlled delivery of the implant 260 to the target canal.

In addition to the central opening, the barrel covers 226 may also feature barrel cover stabilizing apertures 228. These apertures can be distinct from the central aperture and are designed to accommodate the stabilizing plunger 240. The stabilizing plunger 240 may pass through the barrel 210 by way of these stabilizing apertures 228. If the stabilizing plunger 240 is composed of at least two elongated members, there may be a corresponding number of barrel cover stabilizing apertures 228 to guide each member, thereby providing stability during operation.

An aperture is provided at the engagement end of the barrel 110, 210, 310, 410, designed to allow the seamless passage of the implant 160, 260 into the punctal opening when using the device. The circumferential border of the aperture may include openings corresponding with the axial gaps, allowing the plunger 130 to pass at least partially through the aperture during administration of the implant 160, 260. In some embodiments, the engagement end of the barrel 110, 210, 310, 410 may include a tip 150, 250, 350 that is tapered, aiding in gentle and atraumatic insertion into the delicate punctal tissues. Axial gaps may be provided through the tip, allowing the protruding guides of the plunger 130 to continue traveling through the tip. In at least one embodiment, the tip 150, 250, 350 may be constructed using an at least partially flexible material, allowing the tip 150, 250, 350 to deform as the implant 160, 260 is passed through via the plunger 130.

A loading section 214, 314, 414 and loading platform 415 may be located near the engagement end. The loading platform 415 may serve as a designated area for loading the implant 160, 260 into the insertion device. In some embodiments, the loading platform 415 may be oriented more inward along the barrel 210, 310, 410 from the engagement end, promoting easy access and secure placement of the implant 260 into the barrel 210, 310, 410 prior to operation.

The loading platform 415 may include a portion of material extending outwardly from the tubular surface of the barrel 410. The loading platform 415 may advantageously allow increased flexibility with orienting the implant with respect to the loading interface. For example, if an implant is positioned near the loading interface at an imperfect angle, the loading platform 415 may assist with guiding the implant into the barrel 410 via the loading interface and reducing the chance of it instead falling or dropping. In some embodiments, multiple loading platforms 415 may be provided, for example, extending from opposing edges in opposite directions, which may further assist with loading the implant into the barrel 410.

Distal to the engagement end of the barrel 110, 210, 310, 410, an action end may be held and engaged by a user operating an insertion device enabled by this disclosure. Flanges may be included at the action end of the barrel 110, 210, 310, 410, which may extend outwardly from the material of the barrel 110, 210, 310, 410. The flanges may provide a surface for the practitioner to grip, facilitating controlled and stable handling of the device during insertion. The flanges may also act as depth gauges, helping the practitioner to insert the implant 160, 260 to the desired depth within a canal. The flanges may also advantageously contribute to the overall stability of the device during the insertion process, reducing the risk of slippage or misalignment. In some embodiments, the design of the flanges can be customized to suit different implant 160, 260 types and sizes, ensuring optimal performance for various clinical applications.

In some embodiments, an additional stabilizer gap may be provided near the action end to correspond with a stabilizer tab of a plunger. Complimentary to the axial gaps and protruding guides near the engagement end of the barrel the stabilizer gap may help maintain linear and accurate movement of a plunger including a stabilizer tab as it passes through the barrel.

Those of skill in the art will appreciate that a barrel enabled by this disclosure may be adapted to different implant types and sizes, with the customizable flanges, contributing to the device being versatile for various clinical applications. The incorporation of features like the axial gaps, loading interface, loading platform, and flanges advantageously provides a precise and controlled implant delivery mechanism that enhances the clinical effectiveness of the orifice insertion device, improving patient outcomes and making it a valuable device for managing tear drainage disorders, delivery of medication or other medical devices, and improving patient quality of life.

An embodiment including a barrel loading interface 214, 314, 414 will now be discussed in greater detail. FIGS. 6-8 and 12 highlight examples of the barrel loading interface 214, 314, 414, which may also be shown in other figures. In one embodiment, the barrel 210, 310, 410 may include a loading interface between the engagement end and the action end, typically oriented closer to the engagement end, into which an implant 260 may be inserted prior to delivering the implant 260 to a canal of a patient. The implant 260 may be guided into the loading interface via a loading platform 415. Optionally, one or more loading doors may be reversibly closed to secure the loading platform 415 and any contents loaded to the apparatus via the loading platform 415.

The loading platform 415 may be located near the engagement end of the barrel 210, 310, 410, typically at the same position as the loading interface, to facilitate loading the implant 160, 260 into the barrel 210, 310, 410 by guiding the implant 260 into the loading interface. The loading platform 415 may prevent accidental movement or dislodgement of the implant 260 prior to being received by the barrel 210, 310, 410 via the loading interface, particularly in situations where space is limited, or the practitioner's dexterity is challenged.

In embodiments in which the loading platform is moveable, the loading platform 415 may operate as a loading door. In this configuration, the loading doors may pivot about an edge operatively in contact with the barrel 410. For example, a thin length of material may provide additional flexibility that promotes bending and movement. In another embodiment, a hinge may be provided. The loading platform 415 may seamlessly integrate with the barrel’s structure, maintaining the structural integrity of the barrel 410.

The tip will now be discussed in greater detail. FIGS. 1-4, 6-8, 11, and 13-15 highlight examples of the tip, which may also be shown in other figures. The accompanying drawings highlight examples of the tip. The tip 150, 250, 350, when included, may provide a point of contact between an orifice insertion device enabled by this disclosure and the patient's punctal opening. In other embodiments, the barrel 110, 210, 310, 410 may contact the punctal opening directly without additionally passing through a tip.

In embodiments including a tip, the tip 150, 250, 350 may be designed to securely and seamlessly connect to the engagement end of the barrel 110, 210, 310, 410, ensuring a continuous pathway for the implant 160, 260 delivery. Connections may include threaded installation, pressure, welding, locking ridges, or other connection techniques that would be appreciated by a person of skill in the art after having the benefit of this disclosure. The tip 150, 250, 350 may be constructed using materials similar to the barrel 110, 210, 310, 410, which may include biocompatible materials that are safe for use in the eye and minimize the risk of irritation or adverse reactions.

The tip's shape and size may facilitate smooth and atraumatic insertion into the delicate punctal opening or other orifice. The tip 150, 250, 350 may be tapered or rounded to minimize tissue trauma. The tip 150, 250, 350 may feature an aperture or opening through which the implant 160, 260 may pass during the insertion process. The size and shape of this aperture may be matched to the implant 160, 260 being used. For example, the implant 160, 260 may travel through the engagement end of the barrel 110, 210, 310, 410 and emerge through the tip's aperture to be inserted into the punctal canaliculus. In some embodiments, at least part of the tip 150, 250, 350 may be deformed as the implant 160, 260 passes through its interior and/or aperture. In embodiments including axial gaps, the tip 150, 250, 350 may include axial gaps that correspond with the axial gaps provided by a barrel 110, 210, 310, 410, which may guide the protruding portion 138 of the plunger 130 to remain aligned for accurate placement within the punctal opening within the canal of the patient.

The plunger will now be discussed in greater detail. FIGS. 1-2, 5-7, 9-11, and 13-15 highlight examples of the plunger, which may also be shown in other figures. The plunger 130 may be provided as a length of material that extends from a plunger delivery surface 134, 234 at an engagement end 132, 232, 332 to a distal action end 136, 236, 336. The plunger 130 may be located at least partially within the barrel 110, 210, 310, 410 and can be moved throughout the barrel 110, 210, 310, 410. In some embodiments, the plunger 130 may include a protruding portion 138 at the engagement end that extends beyond the barrel 110, 210, 310, 410 through one or more of the axial gaps, assisting with controlling the motion of the plunger 130 as it passes through the barrel 110, 210, 310, 410.

In some embodiments, the protruding portion 138 of the plunger 130 may extend beyond the barrel 110 via one axial gap. In other embodiments, the protruding portion 138 of the plunger 130 may extend beyond the barrel 110, via multiple axial gaps, for example two opposed axial gaps. In this example, a protruding end of the plunger 130 can be configured as a “Tee” to be located inside the barrel 110, and pass through and be guided by the axial gaps. In some embodiments, the plunger 130 may include a stabilizer tab that can correspond with a stabilizer gap of the barrel 110, through which the plunger 130 may pass. The plunger surface and the interior of the barrel may be designed to facilitate smooth, low-friction movement, reducing the risk of damage to the implant during delivery.

The plunger 130 may be operated to push the implant 160, 260 from the barrel 110, 210, 310, 410 into the targeted punctal opening or other orifice. Typically, the plunger 130 may function as a rod or piston-like component that moves within the barrel 110, 210, 310, 410, applying controlled force to the implant 160, 260. The plunger 130 may be designed to facilitate precise control over the implant 160, 260 movement, enabling accurate placement within the delicate punctal canal or other canal.

As discussed above, the plunger 130 may include a protruding portion 138 that extends beyond the barrel 110, 210, 310, 410 through axial gaps at the engagement end. In certain embodiments, the protruding end of the plunger 130 may be configured as a "Tee" shape to enhance stability and guidance within the barrel 110, 210, 310, 410 having two axial gaps, contributing to precise implant delivery. Those of skill in the art will appreciate additional shapes for the plunger may be provided to correspond with the number and configuration of gaps provided by the barrel 110, 210, 310, 410 through which the plunger may travel. In other embodiments, a flattened pushing surface may be provided to apply even force on the implant 160, 260 and/or surrounding tissue via an increased surface area for even force distribution.

In some embodiments of the insertion device, the plunger may be a multi-part assembly comprising a central plunger 230 and a stabilizing plunger 240. The central plunger 230 may extend longitudinally from a central plunger engagement end 232 to a central plunger action end 236. Likewise, the stabilizing plunger 240 may extend from a stabilizing plunger engagement end 242 to a stabilizing plunger action end 246. A central plunger passthrough 244 may be provided by the stabilizing plunger 240 at its stabilizing plunger action end 246. This configuration allows for distinct actions to be performed by different parts of the plunger mechanism, which may facilitate a controlled and stable delivery of an implant 260. The design enables the separation of the force-applying component from the tissue-stabilizing component, providing a high degree of precision during a procedure.

In these embodiments, the plunger extends longitudinally from a plunger action end 236, 246, where a user may apply an actuation force, to a plunger engagement end 232, 242, where the device interacts with the implant 260 and the patient's tissue. The different components of the plunger 230, 240 may be manipulated independently to perform sequential tasks, such as loading the implant 260 and then engaging the tissue before final delivery.

The central plunger 230 may contact and deliver the implant 260. It may be configured as a rod or piston-like element that travels within the central axis of the barrel 210. The central plunger 230 may receive the actuation force and transfer it to the implant 260, pushing it toward the barrel engagement end 212 and into the canal. The plunger engagement end 232 of the central plunger 230 may be shaped to effectively engage with the implant 260. In some configurations, the central plunger 230 may comprise a plunger delivery surface designed to distribute the actuation force across an increased surface area of the implant 260, which may reduce the risk of damage to the implant during delivery.

The movement of the central plunger 230 may be guided by barrel covers 226 located at the barrel action end 216 and barrel engagement end 212. These barrel covers 226 may feature a barrel cover central aperture 227, which is an opening sized and positioned to allow the central plunger 230 to pass through. This arrangement helps ensure the central plunger 230 remains properly aligned along the central axis of the barrel 210 as it is advanced and retracted.

The stabilizing plunger 240 may operate alongside the central plunger 230 to engage tissue adjacent to the orifice or canal to provide stability during the delivery of the implant 260. By stabilizing the surrounding area, it may prevent the tissue from moving as the device is operated, ensuring the implant 260 is placed accurately. Furthermore, the stabilizing plunger 240 may be used to selectively configure the depth at which the implant 260 is delivered into the canal. By acting as a physical stop against the tissue surface, it can effectively limit the travel of the device and ensure a consistent and repeatable placement depth. In some embodiments, the stabilizing plunger 240 may comprise at least two elongated stabilizing plunger 240 members.

Similar to the central plunger 230, the stabilizing plunger 240 may also pass through the barrel covers 226. The barrel covers 226 may be equipped with barrel cover stabilizing apertures 228, which are separate openings designed to guide the elongated members of the stabilizing plunger 240. These apertures ensure the stabilizing plunger 240 moves parallel to the central plunger 230 and maintains its position relative to the barrel 210.

To enhance the function of the stabilizing plunger 240, stabilizer feet 248 may be installed at its plunger engagement end 242. These feet 248 are the components that contact and selectively abut the tissue adjacent to the orifice to provide a stable platform against the tissue surface. The stabilizer feet 248 may be designed with a surface area that distributes pressure evenly on the tissue, preventing trauma while providing a firm hold. Their shape may vary, and they could be configured as two distinct feet or as a single semicircular or circular foot.

The installation of stabilizer feet 248 to the stabilizing plunger 240 may provide a contact point for setting the implant 260 depth. In some embodiments, the stabilizer feet 248 may provide similar functionality as the protruding portion 138 described above. For example, the physical relationship between a plunger delivery surface 234 at the end of the central plunger 230 pushing the implant 260 and the surface of the stabilizer feet 248 resting on the tissue can be manufactured to achieve a desired, repeatable placement depth for the implant 160, 260. In the embodiment with stabilizer feet 248, this feature advantageously allows the implant 260 to be placed deep within the canal, level with the surface, or slightly protruding from the canal, depending on the design.

The stabilizing plunger 240 may be capable of moving independently from the central plunger 230. This separation of function allows for a multi-stage operation that can enhance the precision and ease of use of the device. For instance, in a non pre-loaded version, the independent movement may allow for loading of the implant 260 into a barrel loading section 214 without disrupting or prematurely depressing the stabilizing plunger 240 and any attached stabilizer feet 248. The central plunger 230 can be advanced independently to make contact with the loaded implant 260 and move it into a ready position near the tip.

The implant will now be discussed in greater detail. The implant may be a small object designed to be inserted into a canal of a user, for example via the punctal opening or other orifice. In one example, the implants may manage conditions related to drainage, for example tear drainage related to dry eye syndrome or other conditions limiting nominal function of an anatomical canal. In additional embodiments, the implants may manage drug delivery, including slow continuous or pulsed drug delivery.

As will be appreciated by those of skill in the art, the specific type of implant used will depend on the procedure to be performed and/or desired therapeutic outcome. In one example, the implant may be a punctal implant being small, biocompatible, and designed to partially or completely block the punctal opening to reduce tear drainage and increase the eye's moisture levels. Another example of an implant may include a drug-eluting implant designed to release medication over time, providing targeted treatment to the ocular surface or surrounding tissues. Drug-eluting implants may deliver anti-inflammatory drugs, antibiotics, or other medications to a target location. Additional examples of implants may include, without limitation, stents, plugs, and other implant types that would be apparent to a person of skill in the art after having the benefit of this disclosure.

Implants, for example for punctal use, may be made from biocompatible materials that are well-tolerated by the body and minimize the risk of inflammation or rejection, for example, silicone, hydrogel, collagen, and/or other materials that would be apparent to a person of skill in the art after having the benefit of this disclosure. The implant may be sized and shaped to fit comfortably within the punctal opening and canaliculus without causing discomfort or obstruction. The shape of an implant may vary depending on the specific type of implant and its intended function. The implant may be designed to be comfortable and minimally noticeable once in place.

In an alternative embodiment, an insertion device enabled by this disclosure may be configured to operate via a compression or squeeze mechanism to generate the actuation force. Instead of a direct linear push applied to a plunger action end, a user may be able to squeeze a portion of the barrel or an associated handle assembly. This compressive force may be translated into the longitudinal movement of the plunger within the barrel to deliver the implant. The innovative functions at the barrel engagement end, such as the interaction of the plunger with the implant and surrounding tissue, may be maintained in this configuration.

In this configuration, the barrel may be adapted to include flexible sections or may be integrated with articulated handles that a user can compress. The plunger, while still extending longitudinally, may be operatively connected to an internal linkage system. Despite the different actuation method, the plunger may still travel through the barrel’s central channel. Furthermore, a protruding portion of the plunger at the plunger engagement end may still be guided by a barrel axial gap, which may help to ensure stable and aligned movement during the withdrawal of the barrel and/or tip while performing the implant delivery operation.

In operation, a method may be provided for implant or other payload delivery to a canal of a patient or other subject. Those of skill in the art will appreciate that the following methods are provided to illustrate some embodiments enabled by the disclosure and should not be viewed as limiting the disclosure to only those methods or aspects. Skilled artisans will appreciate additional methods within the scope and spirit of the disclosure for performing the operations provided by the examples below after having the benefit of this disclosure. Such additional methods are intended to be included by this disclosure.

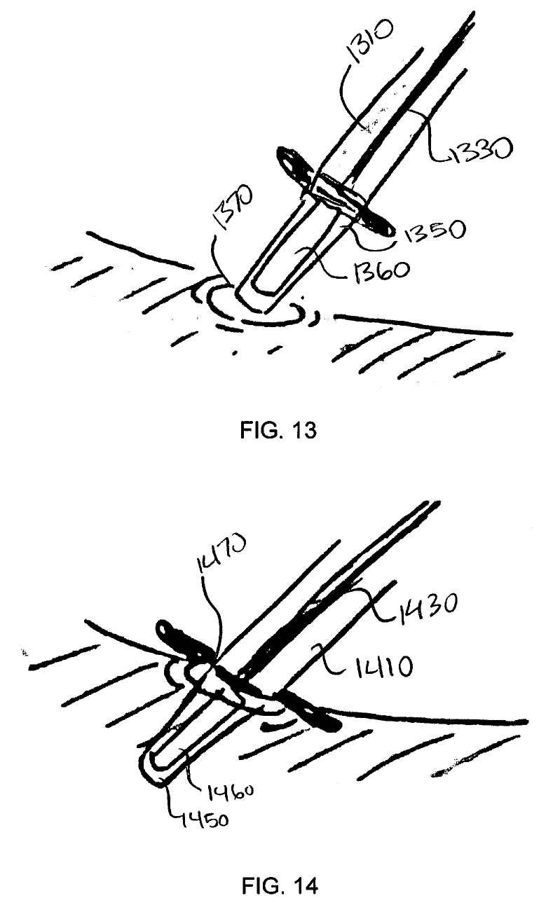

In one example of the operation of an orifice insertion device enabled by this disclosure, an implant may be inserted into a patient canal with improved consistency, predictability, and repeatability. Referring to FIG. 13-16, the process may begin as illustrated in FIG. 13 by the practitioner carefully aligning the device with the patient's punctal canal 1370, 1470, 1570, 1670, which may be aided by a tip 1350, 1450, 1550 located at the engagement end of the barrel 1310, 1410, 1510. Once aligned, the tip 1350, 1450, 1550 of the device may be gently introduced into the punctal opening 1370, 1470, 1570, 1670. By using an insertion device enabled by this disclosure, the device may advantageously engage the punctal opening 1370, 1470, 1570, 1670 without requiring the prior step of dilation. For example, as illustrated in FIG. 14, at least part of the tip 1350, 1450, 1550, 1650 may be inserted into the punctal opening 1370, 1470, 1570, 1670 such to position the implant ready for insertion into the canal 1370, 1470, 1570, 1670.

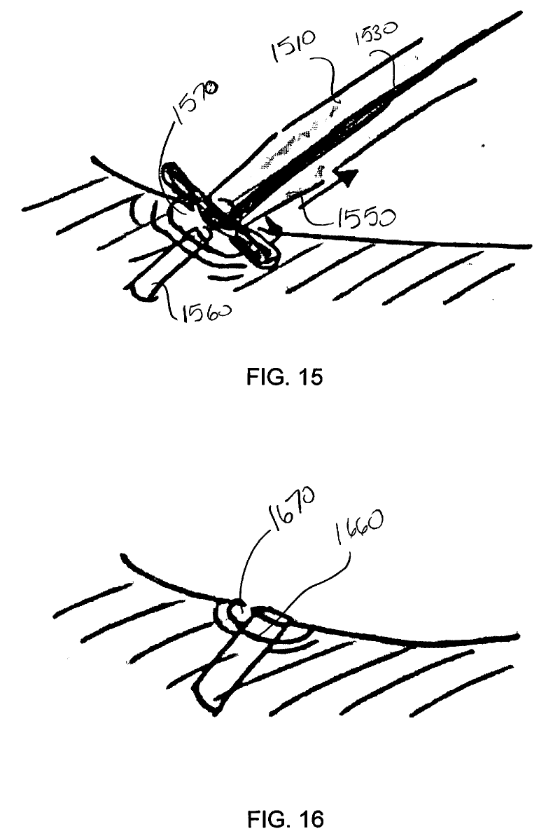

With the tip 1350, 1450, 1550 properly engaged with the canal 1370, 1470, 1570, 1670, the plunger 1330, 1430, 1530 may be further actuated to deliver the implant 1360, 1460, 1560, 1660 into the punctal canaliculus or another canal 1370, 1470, 1570, 1670. The barrel 1310, 1410, 1510, 1610 and the tip 1350, 1450, 1550, 1650 may then be withdrawn from the punctal opening 1370, 1470, 1570, 1670 while the plunger 1330, 1430, 1530, 1630 remains in position to hold the insert 1360, 1460, 1560, 1660 within the canal, as is illustrated in FIG. 15. The remaining aspects of the insertion device may then be withdrawn to leave the insert 1360, 1460, 1560, 1660 within the punctal opening 1370, 1470, 1570, 1670, as illustrated in FIG. 16.

In one embodiment, the depth of implant placement may be controlled by an offset between the external and internal portions of the plunger. This innovative design allows for accurate and repeatable placement at the desired depth, whether deep within the canaliculus, level with the surface, or slightly protruding. Unlike traditional devices that rely on a depressing motion to determine insertion depth, an orifice insertion device enabled by this disclosure installs the implant to a desired depth predictably and consistently.

The plunger's bearing surface, strategically offset from its protrusions, further contributes to precise and repeatable implant placement. This design feature allows practitioners to achieve consistent results, regardless of their experience level or the specific anatomical variations of the patient.

An orifice insertion device enabled by this disclosure may employ a unique retraction mechanism, wherein sidewalls of the device may retract distally, gently revealing the implant while providing slight traction to surrounding tissues as the implant exits the tip. This helps to ensure that the implant is securely positioned within the canaliculus, minimizing the risk of expulsion. In one example, the tip may be constructed from compliant materials that allow for slight expansion during removal. Providing an at least partially deformable tip may enable the practitioner to perform punctal insertion, dilation, and implant delivery in one smooth, approximately continuous motion, streamlining the procedure and enhancing patient comfort.

As the sidewalls of the tip may retract to reveal the implant, the tip may maintain gentle contact with the surrounding tissue. This not only reduces the likelihood of implant expulsion but also helps to minimize discomfort or trauma to the delicate tissues of the punctal opening. This feature may assist with reducing both procedural time and patient anxiety. The ability to perform the procedure ambidextrously from either side of the patient further enhances its versatility and ease of use.

In another example, an illustrative operation of an insertion device enabled by this disclosure, in an embodiment with a plunger that includes a central plunger and a stabilizing plunger, will now be discussed, without limitation. This illustrative operation may begin with the preparation of the device and the implant prior to approaching the patient. Initially, the insertion device, in this case a non pre-loaded version, may be held in preparation to load the implant into the barrel loading section.

The practitioner may then place the implant within a central passage of the barrel via the barrel loading section. Once the implant is situated within the barrel, the next step involves engaging it with the central plunger. This is possible due to the independent movability of the central plunger relative to the stabilizing plunger. This design allows for the implant to be loaded and positioned without disturbing the stabilizer feet at the engagement end of the device.

The practitioner may then gently depress the central plunger, advancing it forward until its plunger engagement end makes contact with the implant. After making contact, the central plunger may be depressed further to advance the implant from the loading area toward the barrel engagement end. This positions the implant so it is ready for insertion and also protects it from premature hydration. At this stage, the central plunger may be visible through the access window of the barrel loading section, indicating the device is advanced and ready for the next step.

With the implant now in a ready position, the practitioner may carefully align the insertion device with the patient's orifice, such as a punctum. The tip of the device, if present, may be gently introduced into the orifice. The device may be inserted into the canal until the stabilizer feet, which are installed at the engagement end of the stabilizing plunger, make contact with the tissue surrounding the orifice. For example, in an ophthalmic procedure, the feet may contact the eyelid tissue surrounding the punctal opening. The contact provides a stabilizing effect on the surrounding tissue, such as the eyelid, holding it in place during the subsequent delivery of the implant.

The stabilizer feet may also function to set the depth of delivery. By providing a firm stop against the outer surface, they create a fixed reference point. The depth at which the implant will be placed is determined by the designed offset between the surface of the stabilizer feet and the end of the central plunger pushing the implant. This allows for a repeatable placement of the implant deep within, level with, or protruding outwardly from the surface as desired.

Once the device is seated and the tissue is stabilized by the stabilizer feet, the practitioner may hold the barrel in place while the plunger is depressed. This action applies the final actuation force to the central plunger, causing it to advance relative to the now-stationary stabilizing plunger.

As the central plunger moves forward, it pushes the implant out of the tip and into the canal as viewed from the point of reference of the barrel of the insertion device. This same operation, viewed from the point of reference of the delivery location, is analogous to holding the implant in position by the plunger and/or stabilizer feet as the remaining components of the barrel and tip are withdrawn from the punctal opening or other orifice. The stabilizer feet continue to hold the surrounding tissues in place, which ensures the implant is delivered to the proper placement and depth without being retracted or following the inserter upon its removal. This controlled movement helps to securely position the implant within the canaliculus, minimizing the risk of expulsion. The stabilizer feet prevent unintended movement or expulsion of the implant during the final delivery motion.

After the implant has been successfully delivered into the canal, the practitioner can begin to withdraw the insertion device. The stabilizing action of the stabilizer feet during delivery helps ensure that the implant remains securely seated within the canal and is not dislodged as the tip and barrel are removed. The device is carefully retracted back out of the orifice, leaving the implant installed at the desired depth. The procedure may be streamlined because the device can potentially perform insertion, dilation, and implant delivery in one smooth motion, without requiring a preliminary dilation step. The entire operation may be performed with improved consistency and predictability.

The device's adaptability to various implant types and sizes, coupled with its user-friendly operation, makes it a valuable tool for addressing a wide range of tear drainage disorders and drug or delivery and placement, medication administration, and delivery of other medical devices. Its ability to achieve consistent and predictable implant placement, regardless of the practitioner's experience level or the patient's unique anatomy, improves upon the prior state of the art. As shown above in the context of managing dry eye and other conditions, an orifice insertion device enabled by this disclosure may facilitate accurate and controlled implant placement, allowing practitioners of various skill levels to deliver optimal therapeutic outcomes, reduce patient symptoms, and improve patient well-being.

While various aspects have been described in the above disclosure, the description of this disclosure is intended to illustrate and not limit the scope of the invention. The invention is defined by the scope of the appended claims and not the illustrations and examples provided in the above disclosure. Skilled artisans will appreciate additional aspects of the invention, which may be realized in alternative embodiments, after having the benefit of the above disclosure. Other aspects, advantages, embodiments, and modifications are within the scope of the following claims.

What is claimed is:

1. An insertion device to deliver an implant to a canal of a patient via an orifice comprising:
a barrel extending longitudinally from a barrel action end to a barrel engagement end;
a plunger extending longitudinally from a plunger action end to a plunger engagement end;
wherein the plunger is configurable to receive the implant to be delivered;
wherein the plunger selectively travels about the barrel upon application of an actuation force to move the implant toward the barrel engagement end;
wherein the implant is selectively pushed by the plunger through the barrel engagement end to be operatively delivered to the canal; and
wherein the implant is deliverable to the canal without requiring preliminary dilation of the orifice.

2. The insertion device of claim 1:
wherein the barrel comprises a barrel axial gap extending longitudinally inward from the barrel engagement end; and
wherein the plunger comprises a protruding portion that at least partially protrudes beyond a barrel circumference via the barrel axial gap.

3. The insertion device of claim 2, wherein the plunger engagement end comprises a plunger delivery surface to distribute the actuation force provided via the plunger to an increased surface area of the implant.

4. The insertion device of claim 3, wherein the plunger delivery surface extends outwardly from the barrel axial gap to selectively abut tissue adjacent to the orifice.

5. The insertion device of claim 3, wherein the plunger delivery surface comprises segments of varied depth to deliver the implant to a selectable depth within the canal comprising:
a first delivery surface depth to engage with the implant such to apply a pushing force to deliver the implant to the canal at the selectable depth; and
a second delivery surface depth to interact with tissue adjacent to the orifice and effectively limit travel of the plunger.

6. The insertion device of claim 1 further comprising a tip operatively installed to the engagement end of the barrel to selectively interface with the orifice during operation, wherein the implant passes through the tip from the barrel.

7. The insertion device of claim 6, wherein the tip comprises a tip axial gap that reversibly expands upon passing of the implant through the tip to permit guided installation of the implant to the canal.

8. The insertion device of claim 1, the barrel further comprising a barrel loading section located on a side of the barrel through which the implant is selectively loaded into the barrel.

9. The insertion device of claim 1, wherein the plunger further comprises:
a central plunger to engage with and selectively deliver the implant; and
a stabilizing plunger capable of moving independently from the central plunger to engage tissue adjacent to the orifice to provide stability during delivery of the implant and selectively configure a depth at which the implant is delivered into the canal.

10. The insertion device of claim 9:
wherein the barrel comprises barrel covers located at the barrel action end and the barrel engagement end;
wherein the central plunger passes through the barrel via the barrel covers through a barrel cover central aperture; and
wherein the stabilizing plunger passes through the barrel via the barrel covers through barrel cover stabilizing apertures.

11. The insertion device of claim 9, wherein stabilizer feet are installed to the stabilizing plunger at the plunger engagement end.

12. The insertion device of claim 10, wherein the stabilizing plunger comprises at least two elongated stabilizing plunger members.

13. An insertion device for interacting with an orifice of a body comprising:
a barrel extending longitudinally from a barrel action end to a barrel engagement end and comprising a barrel axial gap extending longitudinally inward from the barrel engagement end;
a tip operatively installed to the barrel engagement end;
a plunger extending longitudinally from a plunger action end to a plunger engagement end and comprising a protruding portion that at least partially protrudes beyond a barrel circumference via the barrel axial gap;
wherein the plunger is configurable to receive the implant to be delivered;
wherein the plunger engagement end comprises a plunger delivery surface to distribute the actuation force provided via the plunger to an increased surface area of the implant;
wherein the plunger selectively travels about the barrel upon application of the actuation force to move the implant toward the barrel engagement end; and
wherein the implant is selectively pushed by the plunger through the barrel engagement end and the tip to be operatively delivered to a canal of a patient via the orifice.

14. The insertion device of claim 13, wherein the plunger delivery surface extends outwardly from the barrel axial gap to selectively abut tissue adjacent to the orifice.

15. The insertion device of claim 13, wherein the tip selectively interfaces with the orifice during operation, and wherein the implant is deliverable to the canal without requiring preliminary dilation of the orifice.

16. A insertion device to deliver an implant to a canal of a patient via an orifice comprising:
a barrel extending longitudinally from a barrel action end to a barrel engagement end;
a plunger extending longitudinally from a plunger action end to a plunger engagement end, the plunger further comprising:
a central plunger configurable to receive and selectively deliver the implant, and
a stabilizing plunger capable of moving independently from the central plunger to engage tissue adjacent to the canal to provide stability during delivery of the implant and selectively configure a depth at which the implant is delivered;
wherein the plunger selectively travels about the barrel upon application of an actuation force to move the implant toward the barrel engagement end; and
wherein the implant is deliverable to the canal without requiring preliminary dilation of the orifice.

17. The insertion device of claim 16, wherein the central plunger comprises a plunger delivery surface to distribute the actuation force provided via the central plunger to an increased surface area of the implant.

18. The insertion device of claim 16, wherein stabilizer feet are installed to the stabilizing plunger at the plunger engagement end to selectively abut the tissue.

19. The insertion device of claim 16, the barrel further comprising a barrel loading section located on a side of the barrel through which the implant is selectively loaded into the barrel.

20. The insertion device of claim 16, further comprising a tip operatively installed to the barrel engagement end to selectively interface with the orifice, the implant selectively passing through the tip from the barrel.
